(19) United States
(12) Patent Application Publication
(10) Pub. No.: US 2020/0225241 A1
(43) Pub. Date: Jul. 16, 2020

(54) Podocan as a Predictive Biomarker of Cardiovascular Events

(71) Applicant: Randolph Hutter, Fly Creek, NY (US)

(72) Inventor: Randolph Hutter, Fly Creek, NY (US)

(21) Appl. No.: 16/741,739

(22) Filed: Jan. 13, 2020

Related U.S. Application Data
(60) Provisional application No. 62/791,832, filed on Jan. 13, 2019.

(51) Int. Cl. G01N 33/68

(52) U.S. Cl. CPC G01N 33/6803; G01N 2333/47; G01N 2800/50; G01N 2800/324; G01N 2800/56; G01N 2800/60

(57) ABSTRACT

Use of blood podocan levels as a biomarker for assessing the probability of experiencing adverse cardiovascular events. The subject patient may be someone who has known CAD or newly diagnosed CAD, and the biomarker is used for secondary prevention. A blood sample is taken from the patient and is assayed for the amount of podocan, which is compared against one or more reference standards. If the measured podocan level meets or exceeds a reference standard, then the test is considered positive for the risk or diagnosis. Podocan could be used in combination with DKK-1 as dual biomarkers predictive of adverse cardiovascular events.

### TECHNICAL FIELD

The invention relates to methods for assessing for the risk of adverse cardiac events in patients using blood biomarkers.

### BACKGROUND

Smooth muscle cell (SMC) migration is a normal process that occurs during tissue repair in response to arterial vascular injury. SMC migration to the site of injury and deposition of extracellular matrix results in the formation of a fibrous cap. Close regulation of SMC migration and proliferation within the arterial intimal space is critical in maintaining a delicate balance between insufficient and excessive plaque repair. Too much suppression of SMC proliferation can weaken the fibrous cap, resulting in plaque vulnerability that underlies the onset of ACS and CVA. On the other hand, too much SMC proliferation and excessive arterial repair causes intimal hyperplasia, which is a hallmark of accelerated CAD, such as the arterial restenosis that can occur post-PCI.

The small leucine-rich repeat (SLR) protein family is an important component of the extracellular matrix. Podocan is a member of this SLR protein family and was found to be a negative regulator of SMC migration and proliferation of smooth muscle cells. See Hutter et al, “Novel small leucine-rich repeat protein podocan is a negative regulator of migration and proliferation of smooth muscle cells, modulates neointima formation, and is expressed in human atheroma” (2013) Circulation 128(22):2351-63. Being expressed in atheromas, podocan likely has an influence on the progression of CAD. In addition, podocan circulates in peripheral blood and has been shown to be a potent inhibitor of SMC activation via inhibition of the canonical Wnt-signaling pathway. These findings suggest that podocan could be a biomarker that is diagnostic or predictive of atherosclerotic plaque rupture events such as myocardial infarction (MI), cerebrovascular accident (CVA), and death. Also, DKK-1 is another circulating Wnt-pathway inhibitor in humans.

In current clinical practice, there is no reliable way to predict who of the patients with newly diagnosed CAD will do well clinically and who will be at high risk for recurrent clinical cardiovascular events. For this reason, current clinical practice pursues a highly reactive approach to monitor for clinical signs and symptoms of recurrent unstable ischemic heart disease such as angina and dyspnea. Given the non-specific nature of such clinical presentations, tremendous resources are directed to performing frequent and repeat clinical testing to rule out the recurrence of active ischemic heart disease. Often, this results in repeat invasive cardiac testing via angiogram driving up health care cost without improving outcomes.

This ineffective and costly reactive approach is further compounded by the fact that all the non-invasive imaging modalities are designed to detect obstructive CAD with high levels of stenosis, while it is the smaller and vulnerable coronary plaques who are the drivers of recurrent MACE and poor outcomes. Thus, there is a strong need for a reliable and cost-effective clinical tool to risk stratify CAD patients in low risk and high risk cohorts.

### SUMMARY

Abbreviations used: ACS—acute coronary syndrome; CAD—coronary artery disease; CABG—coronary artery bypass graft surgery; CVA—cerebrovascular accident; MI—myocardial infarction; PCI—percutaneous coronary intervention (e.g. balloon angioplasty, stent implantation, atherectomy, etc.); MACE—major adverse cardiovascular event (e.g. cardiac death, MI, CVA); DKK-1—Dickkopf-1; SMC—smooth muscle cells.

The invention relates to the use of blood podocan levels as a biomarker to assess for the risk of adverse cardiac events in patients. The subject patient may be experiencing a variety of different clinical scenarios. In some embodiments, the subject patient is someone who is presenting with clinical chest pain syndrome. In some embodiments, the subject patient is someone who is selected to receive cardiac catheterization with angiography. In some embodiments, the patient is someone who has known stable CAD. In some embodiments, the subject patient is someone with known CAD and who has previously received a PCI procedure.

A blood sample is taken from the patient. The blood sample (or its serum or plasma component) is assayed for the amount of podocan. The amount of podocan is compared against one or more reference standards. If the measured podocan level meets or exceeds a reference standard, then the test is considered positive for the risk or diagnosis. In cases of more than one reference standard, the multiple reference standards may indicate different degrees of probability for the risk or diagnosis. In some embodiments, at least one of the reference standards is associated with a relative risk that is in the range of 1.1 to 3.5; and in some cases, in the range of 1.1 to 3.0; and in some cases, a relative risk that is greater than 1.5; and in some cases, a relative risk that is greater than 2.0. In some embodiments, the numeric value of at least one reference standard is in the range of zero to 0.5 ng/mL; in some cases, in the range of zero to 0.3 ng/mL; in some cases, in the range of zero to 0.1 ng/mL; and in some cases, the value of the reference standard is zero.

The invention uses the patient's blood podocan levels (serum or plasma) as a biomarker. The podocan level may be used to assess the risk probability that the subject patient will experience certain adverse cardiovascular events, which will vary depending on the clinical scenario. In some embodiments, the podocan level is used to assess the risk of the patient experiencing a MACE within a near-term timeframe. In some embodiments, the podocan level is used to assess the risk of the patient experiencing restenosis of an artery that has been treated with PCI.

The podocan level may be used as a diagnostic tool to assess the probability that the subject patient is experiencing certain adverse cardiovascular events, which will vary depending on the clinical scenario. In some embodiments, the podocan level is used to determine whether the patient has severe CAD or ACS. In some embodiments, the podocan level is used to determine whether the patient is experiencing a MACE. In some embodiments, the podocan level is used to determine whether the patient is experiencing restenosis of an artery that has previously been treated with PCI.

The invention may also be considered a method of treating CAD. After the podocan determination, the patient is treated for CAD. Any of the various types of CAD treatments may be implemented, including medications, surgery, or percutaneous interventions. In this instance, the podocan level may be used as a clinical decision-making tool to guide the treatment decision. In some embodiments, if the podocan level is positive, then the treatment may involve performing a cardiac catheterization procedure with intravascular optical coherence tomography (OCT) or intravascular ultrasound (IVUS), in conjunction with coronary angiography. In some embodiments, the method further comprises performing a PCI procedure on the patient, for example, to target the lesions identified by OCT or IVUS. In some embodiments, if the podocan level is negative, then the treatment involves performing coronary angiography without OCT or IVUS.

In some embodiments, the method of the invention be computer-implemented in one or more steps of the method. In some embodiments, the step of comparing the podocan level against one or more reference standards is computer-implemented. In some embodiments, the method may further comprise generating a report that states the relevant diagnosis or risk assessment (as explained above). In some embodiments, the report further suggests a diagnostic procedure or treatment to be performed on the patient based on the diagnosis or risk assessment. The report may be a physical copy (e.g. paper hardcopy) or electronic (e.g. displayed on a monitor screen). This step of generating a report may be computer-implemented.

In any of the above embodiments of the invention, blood DKK-1 could be used in combination with blood podocan levels (i.e. dual biomarkers) in implementing the invention. In some embodiments, at least one of the reference standards for DKK-1 is associated with a relative risk that is in the range of 1.1 to 4.0; in some cases, in the range of 1.1 to 3.5; and in some cases, in the range of 2.0 to 3.0. In some embodiments, at least one of the reference standards for DKK-1 is associated with a relative risk that is greater than 2.0; and in some cases, a relative risk that is greater than 2.5. In some embodiments, the numeric value of at least one reference standard is in the range of 200-900 pg/ml; in some cases, in the range of 250-750 pg/ml; in some cases, in the range of 250-650 pg/ml; in some cases, in the range of 250-550 pg/ml; in some cases, in the range of 250-450 pg/ml.

When podocan is used in combination with DKK-1 as dual biomarkers, their combined relative risk for their respective reference standards may be in the range of 3.0 to 8.0; in some cases, in the range of 4.0 to 7.0; and in some cases, in the range of 5.0 to 6.5. In some embodiments, their combined relative risk is greater than 3.0; and in some cases, a relative risk that is greater than 4.0.

In some embodiments, the invention is a method for treating a patient for coronary artery disease. The method comprises determining a level of podocan in a blood sample of a patient; comparing the podocan level in the blood sample with one or more reference standards; based on the comparison, generating a report that states a diagnosis or risk of coronary artery disease; and based on the report, treating the patient with medication or a procedure.

In some embodiments, the invention is method for assessing a patient's risk of experiencing a major adverse cardiovascular event, comprising: determining a level of podocan in a blood sample of the patient; comparing the podocan level in the blood sample with one or more reference standards; determining a level of DKK-1 in the blood sample of the patient; comparing the DKK-1 level in the blood sample with one or more reference standards; based on the comparisons, determining the patient's risk of experiencing a major adverse cardiovascular event.

In some embodiments, the invention is a method for assessing in a patient, who has previously received a percutaneous coronary intervention (PCI) procedure, the risk of experiencing a restenosis of a coronary artery that was treated with the PCI, the method comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; and based on the comparisons, determining the patient's risk of experiencing restenosis of the coronary artery.

In some embodiments, the invention is a method for determining a type of coronary artery imaging to be performed in a patient who has been selected to undergo cardiac catheterization with coronary angiography, the method comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; and based on the comparisons, determining the type of coronary artery imaging to be performed on the patient.

In some embodiments, the invention is a method for treating a patient for coronary artery disease, comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; based on the comparisons, performing a cardiac catheterization procedure on the patient for coronary angiography in conjunction with imaging by intravascular optical coherence tomography (OCT) or intravascular ultrasound (IVUS); and guided by the OCT or IVUS images, performing percutaneous coronary intervention procedure on a coronary artery.

In some embodiments, the invention is a method for treating a patient for coronary artery disease, comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; based on the comparisons, selecting or modifying a treatment for the coronary artery disease in the patient.

In some embodiments, the invention is a method for treating a patient for coronary artery disease, comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; based on the comparisons, generating a report that states a diagnosis or risk of coronary artery disease; and based on the report, treating the patient with medication or a procedure.

In some embodiments, the invention is a method for assessing a patient's risk of experiencing a major adverse cardiovascular event, comprising: determining a level of podocan in a blood sample of the patient; comparing the podocan level in the blood sample with one or more reference standards; determining a level of DKK-1 in the blood sample of the patient; comparing the DKK-1 level in the blood sample with one or more reference standards; based on the comparisons, determining the patient's risk of experiencing a major adverse cardiovascular event.

In some embodiments, the invention is a method for assessing in a patient, who has previously received a percutaneous coronary intervention (PCI) procedure, the risk of experiencing a restenosis of a coronary artery that was treated with the PCI, the method comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; based on the comparisons, determining the patient's risk of experiencing restenosis of the coronary artery.

In some embodiments, the invention is a method for determining a type of coronary artery imaging to be performed in a patient who has been selected to undergo cardiac catheterization with coronary angiography, the method comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; based on the comparisons, determining the type of coronary artery imaging to be performed on the patient.

In some embodiments, the invention is a method for treating a patient for coronary artery disease, comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; based on the comparisons, performing a cardiac catheterization procedure on the patient for coronary angiography in conjunction with imaging by intravascular optical coherence tomography (OCT) or intravascular ultrasound (IVUS); guided by the OCT or IVUS images, performing percutaneous coronary intervention procedure on a coronary artery.

In some embodiments, the invention is a method for treating a patient for coronary artery disease, comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; based on the comparisons, selecting or modifying a treatment for the coronary artery disease in the patient.

In some embodiments, the invention is a method for treating a patient for coronary artery disease, comprising: determining a level of podocan in a blood sample of a patient; comparing the podocan level in the blood sample with one or more reference standards; determining a level of DKK-1 in the blood sample of the patient; comparing the DKK-1 level in the blood sample with one or more reference standards; based on the comparisons, generating a report that states a diagnosis or risk of coronary artery disease; based on the report, treating the patient with medication or a procedure.

### BRIEF DESCRIPTION OF THE DRAWINGS

FIG. 1 shows a plot of the product-limit estimates (Kaplan-Meier curves) for MACE-free survival estimate for podocan as a predictive biomarker.

FIG. 2 shows a plot of the product-limit estimates (Kaplan-Meier curves) for MACE-free survival estimate for DKK-1 as a predictive biomarker.

FIG. 3 shows a plot of the product-limit estimates (Kaplan-Meier curves) for MACE-free survival estimate when taking podocan and DKK-1 in combination as dual biomarkers.

FIG. 4 shows a bar graph of the Cox proportional hazards model for podocan as a predictive biomarker of near-term MACE.

FIG. 5 shows a bar graph of the MACE incidence density for podocan and DKK-1 in combination as dual biomarkers predictive of near-term MACE.

### DETAILED DESCRIPTION

A clinical study was performed to find an association between podocan and different stages of subclinical CAD (mild obstruction) and clinically significant CAD (severe obstruction and ACS). This study was designed as a prospective, observational study that enrolled all patients (“all-comers”) who presented to the cardiac catheterization lab for angiography for the evaluation of known or suspected CAD. Based on angiographic results, participants were grouped into one of four diagnostic categories:

| Category | |
|---|---|
| Designation | Diagnosis |
| Dx1 | Normal angiogram, no CAD |
| Dx2 | Mildly obstructive CAD (stenosis degree <75% on any |
| | lesion) |
| Dx3 | Obstructive CAD requiring intervention (stenosis degree |
| | >75% on any lesion), but not associated with an acute |
| | event, such as MI. Most received PCI or CABG. |
| Dx4 | Acute coronary syndrome (culprit lesion with vulnerable |
| | features) that required PCI or CABG. |

For the analyses presented herein, only subjects in groups Dx3 and Dx4 were included representing patients with clinically significant CAD.

Sample Collection. Participants presented to the cardiac catheterization lab as either elective or scheduled cases. Prior to catheterization, a peripheral venous blood sample was obtained via an intravenous access line previously placed for the catheterization procedure. In emergent cases of ACS, blood samples were obtained in the same manner, if possible. In some cases, such as myocardial infarction with ST-elevation, this was not possible because of clinical care priorities. In such cases, the blood samples were obtained within 24 hours after cardiac catheterization. Blood samples were frozen and stored in a −80° C. freezer.

Data Collection. The participants' medical records were reviewed to collect relevant patient demographics and characteristics (such as age, sex, race/ethnicity, high blood pressure, body mass index, obesity, smoking, and diabetes). Also, the baseline angiogram results were obtained, along with the SYNTAX score for the angiogram, which is an angiographic grading scheme to determine the complexity of coronary artery disease. For the Dx3 and Dx4 groups, the collected data included whether PCI was performed in conjunction with the baseline angiogram, and if yes, what type of stent and its location.

Participants were monitored (by review of medical records) up to 24 months post-catheterization for occurrence of (1) death due to cardiovascular cause, (2) CVA, and 3) ACS (unstable angina with angiographically defined culprit lesion or type 1 myocardial infarction). A composite of the first occurrence of any of these three outcomes was defined as a major adverse cardiac event (MACE).

### Results

A cohort of 308 patients with angiographic evidence of CAD was enrolled and followed prospectively for MACE. Blood samples were collected at time of cardiac catheterization to measure podocan, DKK-1, and C-reactive protein (CRP-1) using ELISA kits. The assay used to detect podocan in plasma was sensitive to levels as low as about 0.01 ng/mL. Table 1 shows the baseline demographics and characteristics of the patients (“Hx”=medical history; “PAD”=peripheral artery disease).

TABLE 1

| | Total | | Chronic CAD | | ACS | |
|---|---|---|---|---|---|---|
| | (n = 308) | | (n = 273) | | (n = 35) | |
| Age | 66.5 ± 9.5 | | 67 ± 9 | | 61 ± 11 | |
| Female (%) | 106 | (33) | 90 | (31) | 16 | (46) |
| Hypertension (%) | 282 | (89) | 244 | (89) | 26 | (74) |
| Diabetes (%) | 142 | (44) | 124 | (45) | 11 | (31) |
| Hyperlipidemia (%) | 269 | (87) | 243 | (89) | 26 | (74) |
| Hx of CABG (%) | 64 | (20) | 58 | (21) | 3 | (8.5) |
| Hx of PCI (%) | 119 | (37) | 105 | (38) | 9 | (26) |
| PAD (%) | 15 | (5) | 12 | (4) | 3 | (8.5) |

| | | | |
|---|---|---|---|
| LDL (mg/dL) | 96 ± 38 | 94 ± 35 | 112 ± 57 |
| HDL (mg/dL) | 47 ± 16 | 46 ± 14 | 51 ± 28 |
| TG (mg/dL) | 165 ± 160 | 167 ± 167 | 154 ± 89 |
| Creatinine (mg/dL) | 1.07 ± 0.8 | 1.05 ± 0.79 | 1.2 ± 1.2 |
| CRP (mg/dL) | 0.11 ± 0.14 | 0.10 ± 0.13 | 0.18 ± 0.19 |
| LVEF (%) | 49 ± 10 | 49 ± 10 | 48 ± 9.5 |

The Kaplan-Meier method was used to construct survival curves, which were compared using the log-rank test. Cox proportional hazards modeling was used for all univariate and multivariate analyses. The study patients were also evaluated for the following 16 Framingham and other conventional risk factors for CAD: age, gender, hypertension, smoking, diabetes mellitus and HgbA1c, body mass index (BMI), kidney glomerular filtration rate (GFR), left ventricular ejection fraction (LVEF), abnormal lipid profile (total cholesterol, LDL, HDL, and triglycerides), SYNTAX score, C-reactive protein (CRP), and creatinine. Due to the limited number of MACE events (n=34), the proportional hazards regression models were limited in size to only three variables in order to allow for stable parameter estimation. Therefore, the test of whether the relationship of each of the three podocan-DKK1 variables to near-term MACE was independent of the potential confounding variables, was conducted one confounding variable at a time. This resulted in 16 proportional hazard models for each of the three DKK1-podocan variables.

The relationship between the podocan and DKK-1 variables and other continuous variables that were potential confounders was measured using Spearman's correlation. Due to the shape of their distribution, the relationship between the continuous DKK1 and podocan variables and the 15 potential confounding variables was measured non-parametrically. The relationship between podocan and DKK-1 and potential dichotomous confounders (diabetes, hypertension, sex, current smoking) was measured by comparing levels of podocan and DKK1 between levels of the dichotomous variable using the Wilcoxon Rank Sum Test.

Podocan Results: The podocan concentration in peripheral blood was initially measured as nanograms per milliliter (ng/ml) with a lower threshold for detection of 0.01 ng/ml. Because the distribution of measured levels was highly skewed, the podocan levels were converted to the binary form as being present (≥0.01 ng/ml, positive) or being absent (<0.01 ng/ml, negative). As used herein, podocan(−) means <0.01 ng/ml and podocan(+) means ≥0.01 ng/ml.

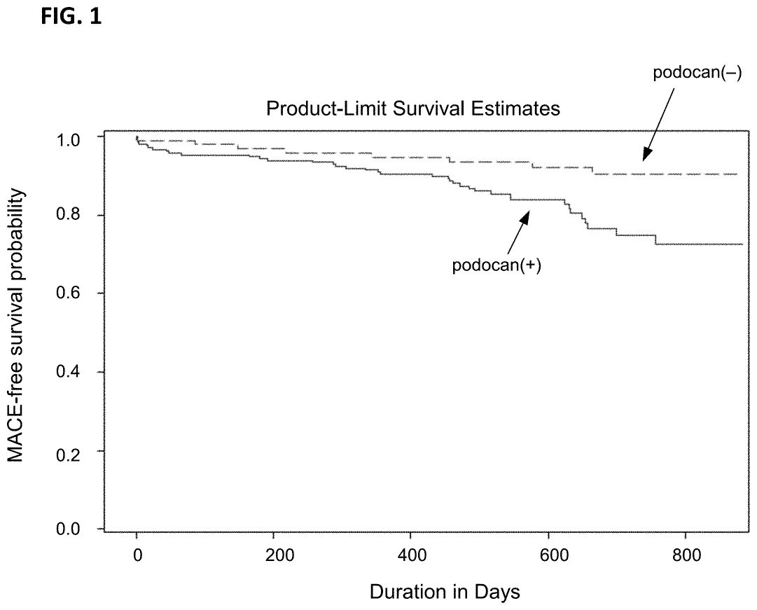

Being podocan(+) is highly predictive of near-term MACE in patients with angiographically defined CAD. FIG. 1 shows a plot of the product-limit estimates (Kaplan-Meier curves) for MACE-free survival estimate for podocan as a predictive biomarker. This plot displays MACE-free survival probability over time for the patients who are podocan(+) versus podocan(−). As seen here, being podocan(+) was associated with an increased rate of near-term MACE. This correlation was significant at the univariate level (HR=2.60, p=0.014). Further, it remained significant in 14 of the 15 two-variable equations where it was paired in turn with each of the 16 confounding variables. The sole exception was for the pairing with CRP, where the probability for podocan was 0.067 (HR=2.23). A higher MACE event rate in podocan(+) patients with becomes increases over time and becomes clear beyond the one-year time point. Podocan was not significantly correlated to any of the conventional risk factors specified above, i.e. podocan was independent thereof.

DKK-1 Results: We also considered the possible role of serum DKK-1 as a predictive biomarker of cardiovascular risk since it has been reported to have a similar Wnt-inhibitory effect like podocan. DKK-1 is a soluble Wnt-pathway inhibitor that shows increased expression in advanced atherosclerotic plaques. Serum DKK-1 was found to be an independent predictor of long-term MACE in patients with ACS. See Lin Wang et al, “Dickkopf-1 as a Novel Predictor Is Associated with Risk Stratification by GRACE Risk Scores for Predictive Value in Patients with Acute Coronary Syndrome: A Retrospective Research” (January 2013) PLoS ONE 8(1):e54731. This study, however, was of a retrospective registry design and did not contain prospective observational clinical data.

The DKK-1 concentration in peripheral blood was initially measured as picograms per milliliter (pg/ml). The results were dichotomized by dividing the group at the median of 362 pg/ml. Thus, designating this as the threshold, the DKK-1 levels were converted to binary form as being high (>362 pg/ml) or low (≤362 pg/ml). Thus, as used herein, “high DKK” means >362 pg/ml; and “low DKK means ≤362 pg/ml.

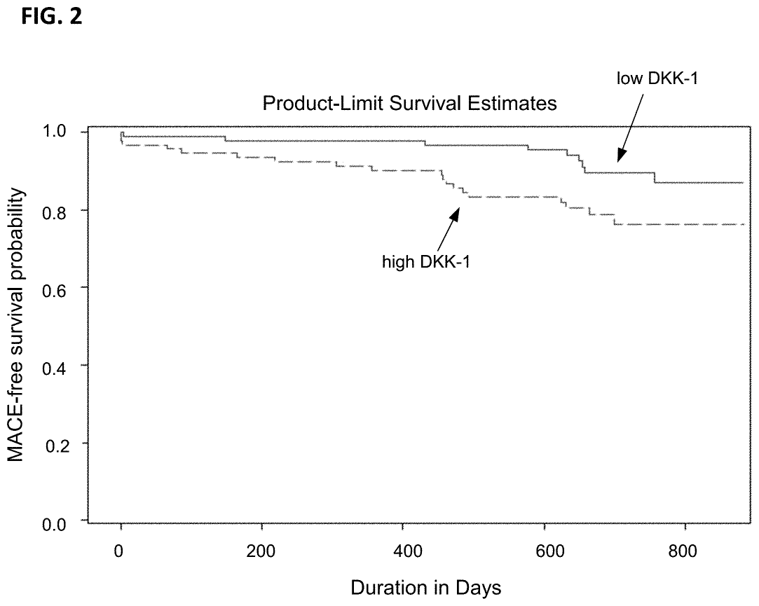

FIG. 2 shows a plot of the product-limit estimates (Kaplan-Meier curves) for MACE-free survival estimate for DKK-1 as a predictive biomarker. This plot displays MACE-free survival probability over time for the patients who are high DKK versus low DKK. As seen here, high DKK was associated with an increased rate of subsequent MACE at the univariate level (HR=2.44, p=0.028). When paired in turn with each of the 15 variables from the list of potential confounders, those with high DKK remained significant in 12 of these equations and was near significant in the other three. These other three equations were creatinine (HR for high DKK1=2.21, p=0.055), ejection fraction (HR for high DKK=2.07, p=0.078), and age (HR for high DKK=2.22, p=0.052). As with podocan, DKK-1 was not significantly correlated to any of the other conventional, confounding risk factors specified above, i.e. DKK-1 was independent thereof.

Podocan/DKK-1 Dual Biomarker: The utility of the combination of podocan and DKK-1 as predictive biomarkers was also analyzed as a composite result. For this purpose, the results were put into three categories: HH (“high-high”) means podocan(+) and high DKK; EO (“either) means podocan(+) and low DKK, or podocan(−) and high DKK; and LL (“low-low”) means podocan(−) and low DKK. Out of 308 patients enrolled in this prospective outcome cohort study, 132 patients (43%) were in the HH category, 43 patients (14%) were in the LL category, and 135 patients (43%) were in the intermediate risk EO category (out of those 81 were podocan(+) and 54 were high DKK). Applying both podocan and DKK-1 in combination had a synergistic effect on predictive value.

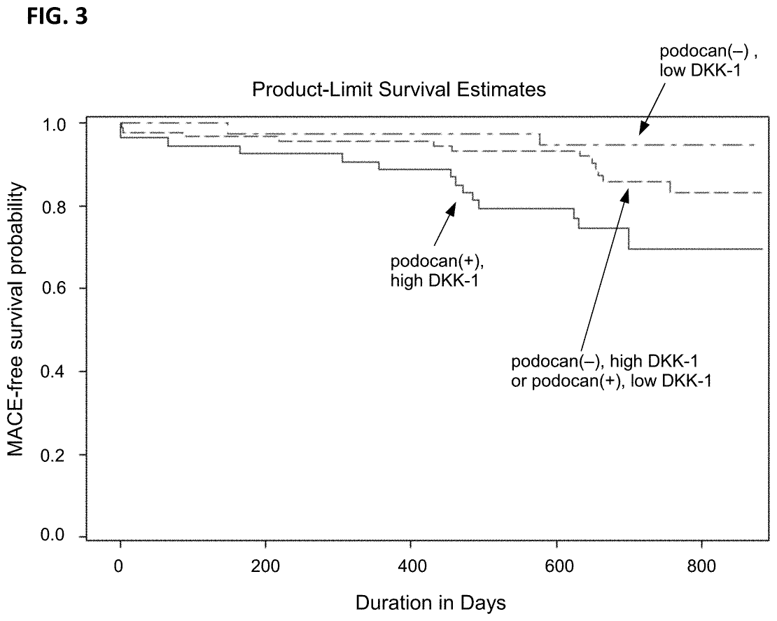

The podocan/DKK-1 composite was strongly related to subsequent MACE. FIG. 3 shows a plot of the product-limit estimates (Kaplan-Meier curves) for MACE-free survival estimate for the dual biomarker. The HH group remained at significantly higher risk for MACE versus the LL group in all equations pairing the three-level variable in turn with each of the 15 potential confounders. Risk for MACE in the HH group also remained significantly elevated over the EO group in 13 of these 15 equations. The two variables which attenuated the increased risk of HH over EO were creatinine (HR for HH versus EO=2.08, p=0.071), and ejection fraction (HR for HH versus EO=1.95, p=0.100).

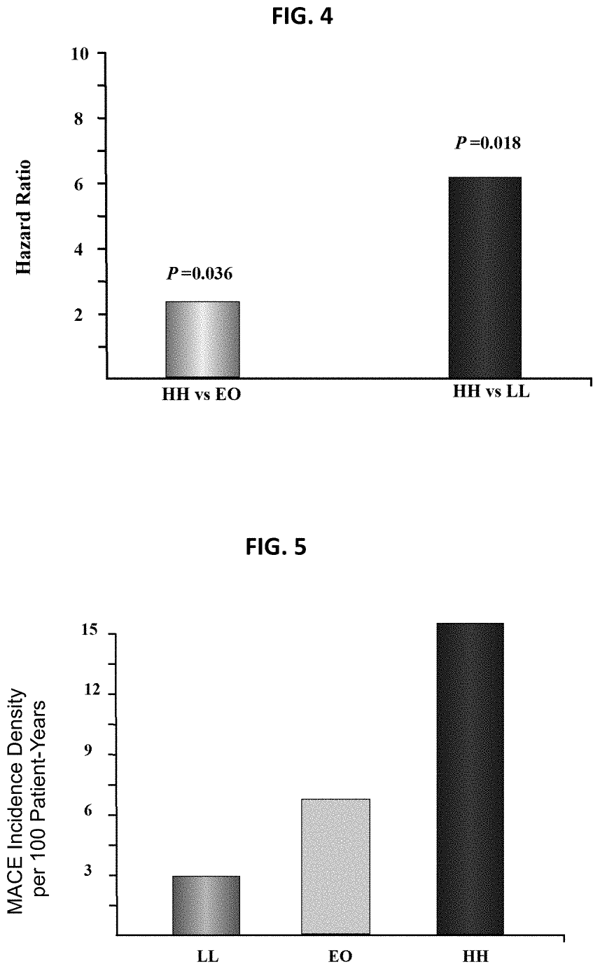

FIG. 4 shows a bar graph comparing the hazard ratio for MACE as expressed on y-axis. The left bar shows patients with the “HH” profile relative to patients with the “EO” profile. This HH/EO hazard ratio was 2.29 (p=0.036). The right bar shows patients with the “HH” profile relative to patients with the “LL” profile. This HH/LL hazard ratio was 5.95 (p=0.018). This shows that the podocan-DKK1 composite biomarker is strongly correlated to near-term MACE. However, because of the limited statistical power, the EO group did not have a statistically significant elevated risk versus the LL group (HR=2.6, p=0.210).

FIG. 5 shows a bar graph of the MACE incidence density for podocan and DKK-1 in combination as dual biomarkers predictive of near-term MACE. Due to the differential follow-up time, risk levels are expressed as incidence density instead of cumulative incidence. The bar graphs compare MACE incidence density on the y-axis as MACE events per 100 patient years observed. The left bar shows patients with the “LL” profile (3/100 person years), i.e. podocan(−) and low DKK-1 levels.

The MACE incidence density for podocan(+) and low DKK was 7.0/100 patient years, whereas the MACE incidence density for podocan(−) and high DKK was 7.6/100 patient years. The similarity of these two incidence densities led to the decision to combine these two groups into the single “EO” group, which denoted intermediate risk. The middle bar shows patients with the “EO” profile (7.0/100 person years), i.e. podocan(−) and high DKK, or podocan(+) and low DKK. The right bar shows patients with the “HH” profile (15.8/100 person years), i.e. podocan(+) and high DKK. As seen here, there is a dose-response-like relationship for biomarker presence and incidence densities.

When expressed in a more tangible cumulative event rate, MACE risk in HH patients with CAD is 18.5% versus 3.4% in LL patients with a mean observation time of around 14 months. Thus, having the HH profile is a very powerful synergistic effect for the dual biomarkers and puts a patient with CAD at nearly six times the risk of MACE compared to LL patients.

Conclusion: These findings suggest that podocan levels (alone or in combination with DKK-1) can be an indicator of vulnerable plaques and of increased plaque rupture risk in patients with established CAD. From a mechanistic point-of-view, it can be hypothesized that the presence of podocan causes oversuppression of SMC proliferation, leading to weakening of the fibrous cap on the plaque and increased risk of death, MI, and CVA (from plaque rupture events in atherosclerotic plaques of the coronary arteries and the aortic arch). The study results are also remarkable for finding that both biomarkers have full independency from traditional Framingham cardiac risk factors, hence opening a new window to personalized risk stratification for patients with CAD. The conventional Framingham risk factor paradigm only functions well in the primary prevention CAD cohort and is not useful in the secondary prevention population due to the uniformly high presence of cardiovascular risk factors in this population as shown by others and also demonstrated in Table 1 above for this study.

Both the podocan and DKK-1 levels in patients presenting to the cardiac catheterization lab, for non-urgent or emergent clinical chest pain syndrome, were predictive of subsequent MACE over the next 24 months. Because this study measured podocan and DKK-1 levels in subjects presenting to the cardiac catheterization lab, these findings suggest that podocan levels (alone or in combination with DKK-1) may be used to determine the further clinical management and further invasive procedures in high risk patients. In particular, this allows more clinical attention to be focused on the high risk cohort, such as selecting the high risk patient for (in conjunction with the planned coronary angiography) intravascular optical coherence tomography (OCT) or intravascular ultrasound (IVUS) for better imaging of the arteries to identify vulnerable lesions and guiding the PCI procedure. This risk stratification could also allow clinicians to select high risk patients for treatment with costly medical therapies, such as PCSK9 inhibitors or intra-coronary invasive plaque imaging.

The foregoing description and examples have been set forth merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method for assessing a patient's risk of experiencing a major adverse cardiovascular event, comprising:
determining a level of podocan in a blood sample of the patient;
comparing the podocan level in the blood sample with one or more reference standards;
based on the comparison, determining the patient's risk of experiencing a major adverse cardiovascular event.

2. (canceled)

3. The method of claim 1, wherein the reference standard(s) is associated with a relative risk that is in the range of 1.1 to 3.5.

4. The method of claim 3, further comprising:
determining a level of DKK-1 in the blood sample of the patient;
comparing the DKK-1 level in the blood sample with one or more DKK-1 reference standards;
based on the comparisons for podocan and DKK-1 in combination, determining the type of coronary artery imaging to be performed on the patient.

5. The method of claim 4, wherein the DKK-1 reference standard(s) is associated with a relative risk that is in the range of 1.1 to 3.5

6. A method for treating a patient for coronary artery disease, comprising:
determining a level of podocan in a blood sample of a patient;
comparing the podocan level in the blood sample with one or more podocan reference standards;
based on the comparison, performing a cardiac catheterization procedure on the patient for coronary angiography in conjunction with intra-coronary invasive plaque imaging.

7. The method of claim 6, wherein the intra-coronary invasive plaque imaging is intravascular optical coherence tomography (OCT) or intravascular ultrasound (IVUS);

8. The method of claim 6, wherein the podocan reference standard(s) is associated with a relative risk that is greater than 1.5.

9. The method of claim 6, wherein the numeric value of at least one podocan reference standard is in the range of zero to 0.5 ng/mL.

10. The method of claim 6, further comprising:
determining a level of DKK-1 in the blood sample of the patient;
comparing the DKK-1 level in the blood sample with one or more DKK-1 reference standards;
based on the comparisons for podocan and DKK-1 in combination, determining the type of coronary artery imaging to be performed on the patient.

11. The method of claim 10, wherein the DKK-1 reference standard(s) is associated with a relative risk that is in the range of 2.0 to 3.0.

12. The method of claim 10, wherein the DKK-1 reference standard(s) is associated with a relative risk that is greater than 1.5.

13. The method of claim 10, wherein podocan and DKK-1 are used as dual biomarkers and their combined relative risk for their respective reference standards is in the 4.0 to 7.0.

14. The method of claim 10, wherein podocan and DKK-1 are used as dual biomarkers and their combined relative risk for their respective reference standards is greater than 3.0.

15. The method of claim 10, wherein the numeric value of at least one DKK-1 reference standard is in the range of 250-650 pg/ml.

16. The method of claim 15, wherein the numeric value of at least one podocan reference standard is in the range of zero to 0.5 ng/mL.

17. A method for treating a patient for coronary artery disease, comprising:
determining a level of podocan in a blood sample of a patient;
comparing the podocan level in the blood sample with one or more podocan reference standards;
based on the comparison, selecting or modifying a treatment for the coronary artery disease in the patient.
